(19) United States
(12) Patent Application Publication
(10) Pub. No.: US 2006/0069064 A1
(43) Pub. Date: Mar. 30, 2006

(54) Methods for facilitating recovery of functions of endogenous or implanted or transplanted stem cells using hyaluronic acid

(76) Inventor: Sophia K. Khaldoyanidi, San Diego, CA (US)

Correspondence Address: CATALYST LAW GROUP, APC, 9710 SCRANTON ROAD, SUITE S-170, SAN DIEGO, CA, 92121, US

(21) Appl. No.: 11/269,321

(22) Filed: Nov. 7, 2005

Related U.S. Application Data
(63) Continuation-in-part of application No. PCT/US04/14260, filed on May 7, 2004.
(60) Provisional application No. 60/469,062, filed on May 7, 2003.

(51) Int. Cl. A61K 31/728

(52) U.S. Cl. 514/54

(57) ABSTRACT

Hyaluronic Acid (HA) is an essential component of tissue extracellular matrices that contributes to the architecture of stem cell niches, which determine the fate of stem cells. Decreased levels of HA are found in subjects experiencing a variety of pathological conditions, as well as in subjects receiving a variety of therapeutic interventions, for example, chemotherapy or radiotherapy, to treat pathological conditions. The use of HA to reconstitute a tissue extracellular matrix partially or completely depleted of HA is described. More particularly, described herein is the use of exogenous forms of HA as an adjuvant in the restoration of the local tissue specific stem cell microenvironment to enhance stem cell recovery or engraftment and thus tissue recovery and remodeling following stem cell transplantation or other therapies. The effect of HA on hematopoietic stem cells is illustrative of the invention. Mice having severe bone marrow hypoplasia, and pancytopenia resulting from treatment with 5-fluorouracil recovered more rapidly if treated with HA. Similarly, mice transplanted with hematopoietic stem cells following lethal irradiation exhibited enhanced recovery of peripheral blood cell counts when treated with HA as an adjuvant therapy compared to control mice transplanted with hematopoietic stem cells without adjuvant therapy.

### RELATED APPLICATIONS

This application is a continuation-in-part of the currently pending international patent application PCT/US2004/014260, filed May 7, 2004 and claiming priority to the U.S. provisional patent application No. 60/469,062, filed May 7, 2003, the disclosures of which are incorporated by reference herein in their entirety.

### STATEMENT OF FEDERALLY SPONSORED RESEARCH

This invention was made in part with Government funding under Grant R21 and Grant K18 awarded by the National Institute of Health. The Government may have certain rights in this invention.

### FIELD OF THE INVENTION

This invention relates to medical treatment protocols involving transplantation or implantation of totipotent, pluripotent and multipotent stem cells (SCs). In another aspect it relates to treatment protocols to reconstitute the extracellular matrix that is required for the tissue architecture and functions of SCs and that is damaged as a consequence of the development of or the treatment of pathological conditions.

### BACKGROUND OF THE INVENTION

Tissues and organs of a mammalian organism are built by mature functional cells of different lineages. Mature cells are terminally differentiated cells that are permanently committed to a specific function(s). These mature cells have a limited life span and, therefore, have to be constantly replenished by their corresponding tissue-specific SCs. The current stage of knowledge in biomedical science is that there are three major types of SCs: totipotent (SCs that give rise to both the placenta and the embryo), pluripotent (SCs that give rise to all embryonic lineages, but not to the placenta) and multipotent (SCs that provide cells for specific organs and tissues). Over the past decade multipotent SCs specific for several tissues and organs have been isolated and characterized. For example, hematopoietic SCs provide for blood cells (erythrocytes, platelets, lymphocytes, monocytes, etc); mesenchymal SCs give rise to a connective tissue (stromal cells, osteoblasts, adipocytes, myocytes, chondrocytes, etc); and neuronal SCs build brain. Other multipotent SCs include adult stem cells, pancreatic stem cells, epithelial stem cells, and endothelial stem cells.

Recent developments arising from stem cell research has generated great interest in the already demonstrated and theoretical applications of stem cells to treat a wide variety of medical conditions. For example, in combination with cytotoxic ablative chemotherapy and irradiation hematopoietic stem cells are already used with success to treat a variety of leukemias and lymphomas. Implanted neuronal stem cells from nasal tissue have been used to treat severed spinal cords in an effort to restore function with a measure of success in the form of at least partial restoration of function below the point of severance. It also has been proposed to use neuronal stem cell implants to treat Parkinson's disease, stroke, and Alzheimer's disease. It has been proposed to use neuronal stem cells from a variety of sources, for example cells from the subventricular zone of the forebrain and the subgranular zone of the dentate gyrus of cadavers, for other applications as well.

Mesenchymal stem cells have been implanted in damaged heart tissue resulting from infarcts and after cardiac surgery and substantial restoration of heart function has been observed. Among the proposed applications for mesenchymal stem cells can be mentioned their use to augment local repair or regeneration of bone, cartilage and tendon; to facilitate the engraftment of hematopoietic stem cells following myeloablative therapy; and to treat osteogenesis imperfecta, osteoporosis, osteoarthritis, meniscectomy, and muscular dystrophy.

Laboratory experiments involving the transplantation of pancreatic stem cells in a diabetic strain of mice have alleviated the diabetic condition of the mice. This strongly suggests that pancreatic stem cell transplantation could be an effective treatment of diabetes mellitus in humans.

It is known that the successful transplantation or implantation of stem cells to achieve therapeutic benefit is dependent on many factors and adjuvant therapies are used to improve the success of these procedures. For example, a variety of soluble factors, cytokines and interleukins are used with varying degrees of success in hematopoietic stem cell transplantation and with many attendant, undesirable side effects. Accordingly, there exists a substantial need for additional adjuvant therapies to be employed with stem cell transplantation and implantation procedures to improve the result obtained using such procedures and to reduce the incidence of undesirable side effects.

SCs constitute a very small population (less than 0.01%) of the mammalian organism. However this number of cells is sufficient to constantly produce billions of new mature cells throughout life. The major features of SCs that distinguish them from all other progenitor cells in the body are 1) the ability for self-renewal, and 2) multipotency. Self-renewal can be defined as the ability of SCs to undergo multiple divisions without also undergoing differentiation, thereby retaining the ability to maintain a pool of SCs. Multipotency is the ability of SCs to differentiate into different lineages, e.g. various cell types. Upon differentiation, SCs lose their “sternness”, i.e. they became mature terminally differentiated cells with mortal fate. Once a SC has chosen a differentiation path, it is believed it can never become a SC again. The behavioral choices (self-renewal, proliferation, or differentiation) of a SC are regulated by multiple signals provided by its microenvironmental niche in response to physiological and pathophysiological demands (Schofield R. Biomed Pharmacother (1983) 37:375-380). These microenvironmental structures have been described for various organs, such a bone marrow, brain, pancreas, etc. The cellular composition of such a niche is very heterogeneous and is comprised of cells of different origin (reviewed in Minguell J J, et al. Braz Jmed Biol Research (2000) 33:881-887; Bianco P, Robey P G. J Clin Investig (2000) 105:1663-1668). Over the past decade, the understanding of molecular mechanisms mediating the regulatory signals provided by the cells of the microenvironmental niches has significantly advanced (Heckney J A, et al. PNAS (2002) 99:13061-13066). Soluble and cell surface associated factors and extracellular matrix (ECM) molecules are produced by the cells that compose the niche and contribute to the highly complex structure of the niche (Gupta P, et al. Blood (1998); 92:4641-4651, and reviewed in Verfaillie C. Blood (1998); 92:2609-2612; Chabannon C and Torok-Storb B. Curr Top Microbiol Immunol (1992) 177:123-136; Klein G. Experientia (1995) 51:914-926). While the cellular composition of niches is tissue specific, extracellular matrix molecules (ECM) represent common features of all niches. ECM components, such as collagens, fibronectin, laminin, and hemonectin, were shown to participate in the tissues' regulatory network, whereas the role of numerous other ECM molecules, including hyaluronic acid (hereinafter, HA), is not yet completely understood.

Without being bound by any particular theory, it is believed that HA is a component of ECMs that is essential for tissue homeostasis. Importantly, CD44, a major receptor for HA, is expressed on the surface of SCs including but not limiting to hematopoietic, neuronal, and mesenchymal SCs. In addition, these SCs demonstrate HA binding ability (Khaldoyanidi, unpublished observations). Therefore, it is believed that HA is required for structuring microenvironmental niches to optimally support the ability of SCs to self renew, proliferate and differentiate. HA, a member of the glycosaminoglycan (GAG) family, is a large negatively charged polymer containing multiple copies of the disaccharide N-acetyl-D-glucosamine (GIcNAc) and D-glucuronate (GlcA). HA is present in all organs and tissues and biological fluids of mammalian organisms. It was initially believed that by binding salt and water, HA expands and maintains extracellular space. Later studies demonstrated that, by interacting with a variety of extracellular molecules, such as aggrecan, versican, neurocan, etc., HA participates in local ECM assembly (Fraser J, et al. J Intern Med. (1997) 242:27-33). Identification of receptors that bind HA demonstrated that HA is implicated in the specific receptor-ligand interactions that ultimately influence cell behavior. Thus, it was revealed that HA is involved in the regulation of multiple cell functions, including cell proliferation, migration, cytokine production, and adhesion molecule expression (Brecht, M., et al. Biochem. J. (1986) 239:445-450; Hamann, K. J., et al. J. Immunol. (1995) 154:4073-4080) (Andreutti, D., et al. J. Submicrosc. Cytol. Pathol. (1999) 31:173-177) (Noble, P. W., et al J. Clin. Invest. (1993) 91:2368-2377; Hodge-Dufour, J. et al. J. Immun. (1997) 159:2492-2500; Khaldoyanidi, S., et al. Blood. (1999) 94:940-949) (Oertli, B., et al. J. Immunol. (1998) 161:3431-3437).

While the involvement of HA in normal cell and tumor biology is generally appreciated, little is known about HA contribution to the assembly of microenvironmental niches that support SCs. Using the hematopoietic system as an example, we have previously reported that HA is not a passive structural element of the bone marrow ECM, but a necessary and specific signal-inducing molecule for hematopoiesis (Khaldoyanidi S, et al. Blood (1999) 94:940-949). Specifically, hyaluronidase (HA'ase) treatment of bone marrow cultures inhibits, or even prevents, lymphopoiesis and myelopoiesis, whereas addition of HA to bone marrow cultures enhances lymphopoiesis and myelopoiesis. It appears that HA regulates a decisive step before the commitment of hematopoietic SCs and is required for SC maintenance and self-renewal. With respect to other tissues and organs, HA was found in the central nervous system (CNS) in perineuronal microenvironment in brain (Giarard et al, Histochem J. 1992;24:21-4), as well as in peripheral nervous system where it is required for myelination of growing nerve (Seckel et al, J Neurosci Res 1995;40:318-24). HA was also shown to be essential in the microenvironment for pancreatic Langerhans islets to support insulin release (Velten et al, Biomaterials 1999;20:2161-7). Since HA synthase-2 knockout mice do not survive in utero as embryos, it appears that HA is required for pluripotent SCs (Camenisch et al, J Clin Invest. 2000;106:349-60).

Although HA is essential for many cell functions, it is an unstable molecule. Total-body irradiation sharply decreases the amount of HA in tissues, including in the spleen and bone marrow (Noordegraaf, E. M., et al. Exp. Hematol. (1981) 9:326-331). Degradation of HA or alteration of its synthesis and accumulation can be induced by various other factors, such as UV irradiation or administration of 5-fluorouracil (5-FU), hydrocortisone or other chemicals. (Koshishi, I., et al. Biochim. Biophys. Acta. (1999) 1428:327-333; Schmut, O., Ansari, and A. N., Faulbom, J. Ophthalmic. Res. (1994) 26:340-343) (Young, A. V., et al. Histol. Histopathol. (1994) 9:515-523; Matrosova V., et al. Stem Cells (2004) 22:544-555) (Yaron, M., et al. Arthritis. Rheum. (1977) 20:702-708). In addition to its depletion as a result of such treatments, a low amount of HA in tissues can be associated with pathological developments such as hormonal imbalance, sclerosis, aging, etc (D'avis et al., Biochem J 1997;324:753-60; Engelbrecht-Schnur et al., Exp Eye Res 1997;64:539-43) (Bodo et al., Cell Mol Biol. 1995:41:1039-49) (Lamberg et al., J. Invest. Dermatol. 1986;86:659-67; Matuoka et al., Aging 1989;1:47-54; Schachtschabel et al., Z Gerontol. 1994;27:177-81). Thus, it is believed that disease- and treatment-induced alterations of the amount of HA in tissues leads to an imbalance of microenvironmental homeostasis and, therefore, affects the function of tissue-specific SCs and aggravates pathological development.

In addition to providing specific receptor-mediated regulation of functions in the microenvironment of SCs, HA is essential for three-dimensional structuring of the niche by binding salt and water and by presenting growth factors. It appears that therapeutic interventions that lead to a decreased amount of HA in tissues can also alter the physicochemical structure of the niche. For example, 5-FU (a drug used in chemotherapy) induced bone marrow hypoplasia and its administration correlates with decreased levels of cell-surface associated HA (Matrosova V. et al. Stem Cells, in press), resulting in negative extravascular pressure outside of bone marrow sinusoids (Narayan et al., Exp Hematol. 1994;22:142-148).

Various pathological conditions or treatments can result in the shedding of HA receptors or down-regulation of their gene expression by cells, including stem cells, progenitor cells, mature cells and microenvironmental cells (Matrosova et al, Stem Cells, 2004, 22:544-555). These changes can result in decreased levels of cell surface associated HA and contribute to the development of sequelae. Therefore, it is important to develop improvements to therapies that enhance the anchoring of endogenous or exogenous HA to the cell surface of stem cells, progenitor cells, mature cells and microenvironmental cells in selected tissues and organs.

Chemotherapy is used alone or in conjunction with radiotherapy for the treatment and cure of a large variety of malignancies. The most undesirable consequences of chemotherapy are severe bone marrow aplasia and pancytopenia. The major reason for this is that chemotherapeutic drugs eliminate not only rapidly dividing cancer cells, but also the pool of cycling hematopoietic progenitor cells. Since mature blood cells have a limited life span they have to be constantly replenished by the committed, actively proliferating progenitors that in turn originate from SCs. Thus, the recovery of mature blood cells following chemotherapy requires a prolonged period of time and is generally accompanied by pancytopenia. Obviously this prolonged period of hematopoietic recovery places patients at a greatly increased risk of infection, bleeding and hypoxia and the attendant consequences, up to and including loss of life, in the hospital setting following transplantation.

Engagement of SCs in proliferation is strictly regulated, and this complex process is controlled by a number of soluble factors, including cytokines and interleukins. Soluble factors mediating SC proliferation are well characterized and are divided into two groups: positive regulators (colony stimulating factors (CSF) such as G-CSF, GM-CSF, M-CSF, erythropoietin (Epo), thrombopoietin (Tpo), interleukins (IL), stem cell factor (SCF), and flt-3 ligand (FL)); and negative regulators of SC proliferation (such as TGF-β, TNFα, LIF, MIP-1α and interferons). It is vital to maintain the correct balance between the positive and negative regulators in order to prevent exhaustion of stem cells and maintain the right ratio of proliferating and quiescent cells in the bone marrow, especially under conditions of physiological demand following chemotherapy, radiotherapy or chemoradiotherapy.

Use of recombinant hematopoietic growth factors has promoted the development of cytokine therapy. Thus, G-CSF and GM-CSF are used to shorten the period of neutropenia in cancer patients following chemotherapy. When used in the appropriate setting, Epo ameliorates anemia following chemotherapy and decreases the need for erythrocyte transfusion in those patients. However, some cytokines, in particular G-CSF, give rise to consistent, severe thrombocytopenia in patients and mice (Momin, F., et al. Proceedings of ASCO. (1992) 11:294. (Abstr.); Scheding, S., et al. Brit. J Haematol. (1994) 88:699-705). Thus, the “lineage competition” effect of G-CSF places patients at increased risk of bleeding, besides exhibiting high toxicity and immunogenic activity. In addition, one of the most important concerns about using growth factors, especially in combination with repeated cycles of chemotherapy, is the potential for stem cell exhaustion. The administration of growth factors not only results in an expansion of the committed progenitor compartment, but also in an increased number of quiescent multipotent SCs entering the proliferative state. Engagement of normally quiescent SCs in the cycling places them at increased risk of massive depletion upon repeated courses of proliferation-dependent chemotherapy (reviewed in Moore M, Blood. (1992) 80(1):3-7). Identification of the molecular mechanisms that prevent quiescent stem cells from entering the proliferative state has a significant potential for clinical applications, especially in view of using repeated cycles of proliferation-dependent chemotherapy.

Thus, it has become clear that there is a need for new approaches in improving the recovery of hematopoiesis following chemotherapy.

Another approach used in the clinic to alleviate sequelae of chemo- and radiotherapy is SC transplantation. Transplantation of hematopoietic SCs is generally used to facilitate hematopoietic recovery following high-dose chemotherapy and total-body irradiation. The efficiency of SC transplantation is reflected by the dynamics of the recovery of peripheral blood cells following transplantation. The efficacy of SC transplantation depends on the homing ability of intravenously infused SCs. As used herein, homing of hematopoietic SCs is defined as the ability of hematopoietic SCs to find the bone marrow hematopoietic niche, to lodge within it, and to produce progeny (Tavasolli M, Hardy C. (1990) Blood 76(6):1059-1070; Hardy C, Minguell J. (1993) Scanning Microscopy 7(1):333-341; Hardy C, Megason G. (1996) Hematol Oncol 14:17-27). Therefore, homing is divided into two major phases: extravasation followed by seeding of the bone marrow. According to this definition, a SC arrested on the bone marrow sinusoidal endothelium is not yet considered a homed cell. Similarly, the extravasated SC that has not reached an appropriate hematopoietic niche and has not produced progeny under the conditions of physiological demand cannot be regarded as a homed cell, either. Extravasation is the first multi-step phase in SC homing and involves interaction of SCs with the bone marrow vascular endothelium under the conditions of physiological flow and includes tethering of cells (e.g., rolling), adhesion to the luminal surface of endothelial cells, and diapedesis (e.g., transmigration) across the endothelium. In the seeding phase, which completes the “homing program,” the extravasated SC must be able to migrate through the bone marrow ECM either using its own enzymic activities or by inducing such activities in the surrounding cells. Finally, the homed cell must (i) find the appropriate microenvironment that produces hematopoiesis-supportive factors and (ii) respond by proliferation and self-renewal (Verfaillie, C. Blood. (1998) 92:2609-2612; Turner, M. Stem Cells. (1994) 12:22-29; Quesenberry, P., and Becker, P. Proc. Natl. Acad. Sci. USA. (1998) 95:15155-15157; Hardy, C., Megason, G. Hematol. Oncol. (1996) 14:17-27; Tavassoli, M., Hardy, C. Blood. (1990) 76:1059-1070).

It should be particularly noted that hematopoietic SC homing/engraftment, which involves facilitation of adhesion of hematopoietic SCs in their microenvironment, namely the bone marrow, their proliferation and self-renewal, is to be distinguished from SC mobilization, which involves the release of anchored SCs and stimulation of their migration from bone marrow into the peripheral blood system. Thus, SC homing/engraftment is the opposite of SC mobilization.

While little is known about the molecular mechanisms mediating directed SC migration, a basis for the understanding of SC homing has been created over the past decade, in which a variety of molecules are implicated, including chemokines such as SDF-1 and cell surface molecules such as P and E selectins, VCAM-1, α4β1 and α4β7 integrins, and CD44 (Khaldoyanidi et al., J. Leuk. Biol. 1996;60:579-92; Frenette et al., Proc Natl Acad Sci USA 1998;95:14423-14428; Williams et al., Nature 1991;352:438; Papayannopoulou et al., Proc Natl Acad Sci USA 1995;92:9647). CD44 was originally described as a homing molecule required for the binding of lymphocytes to high endothelial venules (Jalkanen et al., Science 1986;233:556-558). It has been shown that CD44, in addition to selecting, can mediate the rolling of activated lymphocytes on primary endothelial cells (DeGrendlele et al., J Exp Med 1996;183:1119-1130). It has also been demonstrated that CD44 mediates the in vitro adhesion of lymphocytes and hematopoietic progenitors to HA and fibronectin, important components of the bone marrow ECM (Legras et al, Blood 1997;89(6):1905-1914; Verfaillie et al., Blood 1994; 84(6):1802-1811). Finally, the cytoplasmic part of CD44 specifically binds to cytoskeletal proteins such as ankyrin, and the CD44 variant isoform(s) is/are closely associated with the active form of MMP-9, suggesting that CD44 may be involved in SC migration in extracellular space (Bourguignon et al., J Cell Physiol 1998;76(l):206-215).

In line with these observations, we have previously demonstrated that pretreatment of bone marrow cells with HA-binding blocking CD44-specific antibodies results in a reduction in the ability of hematopoietic SCs to repopulate the bone marrow of lethally irradiated recipients, suggesting that CD44 might interfere with hematopoietic SC homing. Furthermore, we had previously demonstrated that CD44 regulates the initial hematopoietic SC-stromal cell interaction, and therefore might be involved in hematopoietic SC seeding (Khaldoyanidi, S., et al. J Leukoc. Biol. (1996) 60:579-592.). Thus, it is believed that CD44/HA pathway is important for regulation of SC-stromal cell and SC-endothelial cell interactions and, therefore, contributes to the regulation of SC homing/engraftment.

Total-body irradiation results in degradation of HA. Furthermore, reconstitution of lethally irradiated bone marrow with syngeneic bone marrow cells results in a secondary relapse in the GAG concentration in the bone marrow and spleen as compared to non-reconstituted mice. The absence of detectable amounts of HA in the reconstituted mice was remarkable, whereas in the non-reconstituted mice a slow recovery of HA was observed (Noordegraaf, E. M., et al. Exp. Hematol. (1981) 9:326-331). In addition, it was shown that irradiation affects the ratio of sulfated versus unsulfated GAGs, which can be essential for normal hematopoiesis. Therefore, a decrease of the amount of HA resulting from irradiation and the infusion of cells can interfere with homing/engraftment of transplanted SCs.

Transplanted SCs have to repopulate irradiated bone marrow and produce committed hematopoietic progenitors in order to replenish the pool of mature terminally differentiated functionally active blood cells. Thus, recovery of the mature blood cell population following transplantation of hematopoietic SCs requires a prolonged period of time and is generally accompanied by pancytopenia. To facilitate proliferation and expansion of the pool of committed progenitors in post-transplant patients, growth factors are used, in particular GM-CSF. However, in addition to stimulating proliferation of the progenitor cells, GM-CSF mobilizes hematopoietic SCs from bone marrow to peripheral blood. This effect of GM-CSF negatively affects long-term reconstitution by multipotent SCs as a result of these cells becoming sensitive to the growth factors upon mobilization. In view of these and other side effects of GM-CSF, such as bone pain, myalgia, fever and erythrema, the use of GM-CSF is not desirable. Similar to using GM-CSF, Canadian Patent Application No.: 2,199,756 uses what the inventor terms “low molecular weight HA” (178,000 daltons to 562,000 daltons molecular weight range and from 200,000 daltons to 300,000 daltons molecular weight range with streptomyces as the source) to exhibit the capacity for mobilization of SCs, including mature and progenitor hematopoietic cells, from tissues to the periphery. However, there is a need for improved compositions and methods that are useful in facilitating the homing activity of stem cells, thereby drawing these cells to their proper in vivo or ex vivo microenvironmental niche and thereby improving the homeostatic equilibrium at these niches.

### SUMMARY OF INVENTION

The present invention provides a method for treating pathological conditions that are associated with decreased levels of HA in tissues and organs comprising administration of an effective amount of HA. The HA can be administered to a subject, thereby improving the overall in vivo microenvironmental niche. Alternatively, the HA can be administered to an ex vivo cell population, tissue or organ, thereby improving the overall microenvironmental niche and/or tissue or organ specific microenvironmental niches.

The HA is preferably used to provide homeostatic equilibrium to the tissues and organs in post-chemotherapy and post-transplant clinical settings.

It is, therefore, an object of this invention to provide alternatives or complements to the use of G-CSF, GM-CSF and Epo to stimulate post-chemo and post-transplant recovery of tissues.

It is therefore an object of this invention to provide improved treatments of chemo- and irradiation-induced sequelae.

It is an object of this invention to provide an improved method for engraftment of SCs following therapy that depletes SCs.

It is an object of this invention to provide methods of improving the homing of stem cells to a microenvironmental niche in subjects suffering from pathological conditions and treatment therapies that affect this homing process. In one aspect the subjects are treated with an in vivo administration of HA to improve the homing of stem cells.

It is a further object of this invention to facilitate stem cell homing to a microenvironmental niche. In one aspect of this invention, the stem cells are endogenous stem cells and the HA is administered to a subject. In a further object of this invention, the stem cells are exogenous and are transplanted or implanted into a subject. In this aspect of the invention the HA can either be administered to the subject, applied to the stem cells while ex vivo, or both. HA can be administered to the subject before, during and/or after the transplant and/or implant of the stem cells.

It is a still further object of this invention to facilitate the retention of homed stem cells within a microenvironmental niche. In one aspect the stem cells are endogenous stem cells and a subject is administered an effective amount of HA to facilitate stem cell retention within the microenvironmental niche. In another aspect, the stem cells are exogenous and the HA is administered to the stem cells ex vivo before transplanting and/or the HA is administered to a subject before, during and/or after the stem cells are transplanted. By administering an effective amount of HA to retain stem cells within a microenvironmental niche, the stem cells can retain their stem cell characteristics, e.g., self-renewal and multipotency, and can break away from and return to the niche as necessary for maintaining homeostasis within the niche.

It is a further object of this invention to provide methods for improving the number and recovery of stem cells in a subject having a depleted stem cell population caused by a pathological conditions or a treatment therapy.

It is a further object of this invention to provide methods for culturing stem cells wherein said methods facilitate the homing activity of the stem cells. In one aspect the stem cells are cultured for transplanting or implanting into a subject. In a further aspect, the stem cells are expanded in culture for transplanting or implanting into one or more subjects.

### BRIEF DESCRIPTION OF FIGURES

FIGS. 1-4 depict the results of experiments designed to demonstrate the effects of HA on the recovery of bone marrow hematopoiesis in mice after chemotherapy.

FIGS. 5-6 depict the results of experiments designed to demonstrate the effects of HA on engraftment of hematopoietic SC and hematopoietic tissue recovery after lethal irradiation.

FIG. 7 illustrated the range of molecular weights for a mixture of HA polymers. Lane 1 is 1,200 to 2,600 kD HA purchased form LifeCord; lane 2 is 600 kD HA purchased from Lifecord (range not provided by vendor); Lane 3 is 15 kD HA purchased form Lifecord; and Lane 4 is the HA used in the current examples and purchased form Sigma Chemicals, St. Louis Mo.

FIG. 8 illustrates up-regulation of cytokines in the presence of HA (right panel) compared to no HA (left panel).

### DETAILED DESCRIPTION

As used herein, the term “hyaluronic acid” means a polymer comprising repeating dimeric units of glucoronic acid and N-acetyl glucosamine. The polymer can comprise any of a number of these repeating units, thereby providing for a variety of molecular weights. Moreover, the HA used in the current invention can comprise a heterogeneous composition of a mixture of HA polymer sizes, thereby resulting in size ranges and average molecular weights for the composition.

In one example embodiment of a heterogeneous composition useful with the current invention, the HA polymer can have a molecular weight range that is between about 2,000,000 daltons to about 15,000 daltons. In a further embodiment, the HA polymer can have a molecular weight range that is between about 1,500,000 daltons to about 100,000 daltons. In a still further embodiment, the HA polymer can have a molecular weight range that is between about 500,000 daltons to about 1,000,000 daltons. In a further embodiment, the HA polymer can have a molecular weight range that is between about 575,000 daltons to about 900,000 daltons.

Alternatively, the heterogeneous composition of HA polymers used in the current invention can be discussed based on average molecular weight of HA polymers in the mixture. In this aspect, the average molecular weights can be any of a wide variety of weights based upon the distribution of polymer molecular weights making the heterogeneous composition. For example, but not limitation, an HA composition having an average molecular weight of 750,000 daltons can apply to any of the above recited ranges based upon the distribution of polymers in the composition.

In a still further aspect, the molecular weight of the polymers comprising a composition of HA used in this invention is a homogeneous composition and again, the molecular weight of the polymers can fall anywhere within the above ranges. For example, but not as a limitation, there can be provided an HA mixture wherein each polymer is about 750,000 daltons. It is preferred that at least 55% of the polymers within a heterogeneous composition are of the same size; more preferably at least 65% of the polymers are of the same size; still more preferably; at least 70% are the same size; still more preferably at least 75% are the same size; still more preferably at least 80% are the same size; still more preferably at least 85% are the same size; still more preferably at least 90% are the same size; still more preferably at least 95% are the same size; and most preferably 100% are the same size.

The HA can be derived from any source, including but not limited to eukaryotic source, prokaryotic source, animal source, mammalian source, fowl source, bacterial source, fungal source, synthetic source, recombinant source or combinations thereof. More specifically, the source of the HA can be from umbilical cord source, nasal source, rooster comb source, streptomyces source, streptococcal source or combinations thereof. With respect to a recombinant source for HA, the source includes any cell line having a recombinant polypeptide that is capable of synthesizing HA, for example recombinant HA synthase polypeptides. In a preferred embodiment the source of the HA is mammalian, and more preferably umbilical cord.

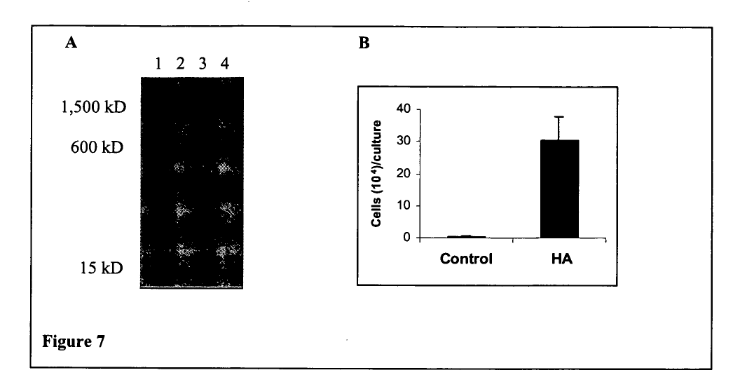

In the current examples, the HA used in the practice of the present invention was purchased from Sigma Chemicals (St Louis, Mo., Cat. No.: H1751). The source of this HA composition is umbilical cord. An aliquot of this HA composition was analyzed to determine the range of molecular weights for the polymers comprising the composition. It was determined that the HA composition used was a heterogeneous composition of HA polymers having an average molecular weight of about 750,000 daltons and a range of molecular weight that is between about 2,000,000 daltons to about 100,000 daltons. (See FIG. 7). For the analysis of the polymer composition the following procedure was performed. A 1% agarose gel was cast (1%; 1× TAE buffer (40 mM Tris acetate, 2 mM EDTA); 40V) using common and well known techniques. Aliquots comprising 7 μg of HA were loaded per lane. (FIG. 7a.) The same amount of the defined size-HA polymers purchased from Lifecord were used as size markers: 1,500 kD (1,200-2,600 kD; lane 1), 600 kD (no range provided by the vendor; lane 2) and 15 kD (lane 3). The HA composition purchased from Sigma for use in the following examples was loaded in lane 4. Staining of HA was performed using Stains All dye (0.005% w/v in ethanol; Fluka). Staining of the gel revealed that the specific HA mixture used herein comprises HA polymers with molecular weight size between about 2,000 kD to about 100 kD (FIG. 7A). This range; however, is not provided herein as a limitation to the invention, only as an illustration of the heterologous nature of the polymer composition.

To evaluate the biological activity of this same specimen the HA composition was added to murine long-term bone marrow cultures (LTBMC). Murine myeloid LTBMC were initiated as follows: Bone marrow cells were obtained from 8-week old BALB/c mice (Harlan, Calif.). Mice were euthanized by a CO₂ overdose; femurs and tibias were dissected and cleaned from muscle tissues. Epiphyses were cut off with scissors at each end of the bone. The contents were flushed out of the bone with PBS- supplemented with 5% FCS using a needle (21G) attached to a 1-ml syringe. To ensure the preparation of a single cell suspension, the cell suspension was aspirated several times through a smaller needle (25G). Freshly isolated bone marrow cells (10⁶ cells/ml) were cultured in Dulbecco modified Eagle medium (DMEM) supplemented with 20% horse serum (StemCell Technologies, Vancouver, Canada) and 10⁻⁶M hydrocortisone (Sigma, St. Louis, Mo.) in 6-well plates at 37° C. in a humid atmosphere containing 5% CO₂. Cultures were fed weekly by changing half of the culture medium. HA (100 μg/ml) was added to LTBMC weekly with feeding media. On week 4, non-adherent cell were harvested from the cultures and counted. The number of non-adherent cells generated in HA-treated LTBMC was significantly higher compared to control (FIG. 7B).

The HA may also be administered in the form of a pharmaceutically acceptable salt, for example, it can be administered as the sodium salt. It is shown herein that the use of HA enhances the recovery of endogenous SCs or engraftment of transplanted or implanted SCs and, thus, tissue recovery and remodeling following stem cell transplantation and other therapies.

The HA is preferably dissolved in an aqueous carrier prior to administration, such as normal saline or any other physiologically acceptable aqueous injectible diluent. Other excipients may include buffers, preservatives, and the like, so long as they are physiologically acceptable. The concentration of the HA solution can be adjusted based on well-known pharmacological principles, but may be between 5 and 500 μg/ml.

Without being bound by any particular theory, it is believed that the beneficial effects that can be obtained using HA result from one or more of the following:
stimulation of the cells of microenvironmental niches to produce soluble factors supportive of SCs;
stimulation of the cells of microenvironmental niches of SCs to express the cell surface factors that support self-renewal of SCs and proliferation and differentiation of committed progenitors;
stimulation of microenvironmental niches of stem cells to produce components of tissue specific ECMs;
stimulation of microenvironmental niches of SCs to produce anti-apoptotic factors;
provision of a direct signal to SCs for their proliferation and self-renewal;
provision of a direct signal to committed progenitor cells for their proliferation;
sequestration and presentation of growth factors and cytokines;
induction of expression and production of endogenous biological activities that stimulate proliferation of committed progenitors without SC exhaustion;
provision of conditions for lodgment of SC in tissues and organs;
anchoring of HA under conditions of disease- or treatment induced decrease in the expression of HA receptors in order to maintain extracellular space and pressure;
directing the migration of cells of any origin;
induction of expression and production of endogenous biological activities with enzymatic properties that mediate SC homing and engraftment;
facilitation of the extravasation of transplanted SC;
facilitation of the seeding/engraftment of bone marrow with intravenously injected SC;
induction of expression of adhesion molecules that mediate SC-endothelial cell and SC-microenvironment cell interactions; and/or
maintenance of the volume and pressure of extracellular sites after therapeutic interventions, including, but not limited to chemotherapy and radiotherapy.
   Treatment Conditions

The invention provides a method to improve/treat the microenvironment niche of SCs in a wide variety of tissues and organs including, but not limited to, bone marrow, brain, pancreas, liver, and skin damaged by therapeutic interventions involving, for example, the use of drugs or ultraviolet, x-ray or other types of radiation.

The invention also provides a method to improve/treat the microenvironment niche of endogenous and transplanted or implanted SCs in tissues and organs such as bone marrow, brain, pancreas, heart, liver, and skin damaged by pathological development of a disease or pathological condition. Examples of such pathological developments are degenerative disorders, primary or subsequent hormonal disorders, aging, pathology of HA synthesis, heart attack and the like.

Accordingly, the practice of the present invention is broadly applicable to treatment of any subject to improve the microenvironmental niche, and in turn facilitate homing of stem cells. Preferably the invention is used for the treatment of a subject that exhibits lower-than-normal (>10% decrease) HA levels as a result of any pathological condition or treatment.

The invention also provides a method for treating and/or improving the interaction of stem cells with the endothelium and in turn improving extravasation of the stem cells towards a microenvironmental niche.

The invention also provides a method to improve/treat the microenvironment niche of SCs in tissues and organs (bone marrow, brain, pancreas, liver, skin, etc) damaged by pathologically expressed HA receptors, such as CD44 and RHAMM (decreased cell surface expression due to shedding or specific down-regulation of gene expression).

It is particularly useful in the treatment of subjects experiencing therapy-induced bone marrow aplasia/hypoplasia, which may be brought on following chemotherapy, irradiation, hormonal therapy, for example, using prednisone, or other therapies known to lead to bone marrow suppression or ablation.

The invention also includes a method for enhancing engraftment of exogenously transplanted SCs comprising administration of a therapeutic amount of a composition comprising HA, or a pharmaceutically acceptable salt thereof, in an aqueous diluent into the peripheral blood or intra-organ or intraperitoneally.

Accordingly, the present invention includes the use of HA to enhance recovery of functions of endogenous or engrafted SCs, including multipotent SCs, e.g., hematopoietic SCs (HSCs), mesenchymal stem cells (MSCs), neuronal stem cells (NSCs), epithelial stem cells (EpSCs), endothelial stem cells (EnSCs), hepatic stem cells (HeSCs), pancreatic stem cells (PSCs), umbilical cord blood SCs and adult stem cells (ASCs), as well as pluripotent, and totipotent SCs. More particularly, it relates to the use of HA to enhance engraftment of the SCs after implantation/transplantation, including the recovery of their “stemness” properties of self-renewal and multipotency and their ability for proliferation and differentiation. In some cases, more than one kind of stem cell can be transplanted or implanted at the same time. For example, MSCs can be implanted with HSCs to support engraftment of the HSCs.

Stem cells useful in the invention for implantation or transplantation purposes can be acquired by isolation procedures well known in the art from any appropriate source, including from the bone marrow, peripheral blood, umbilical cord blood, brain, pancreatic, liver or skin cells, mucosal tissue and the like. These cells can be obtained from the tissue of living donors or cadavers or from stem cells cultured in vitro. Useful multipotent stem cells can also be obtained by causing differentiation of totipotent or pluripotent stem cells or from the corresponding stem cell lines. Stem cells useful in the invention can also be obtained by the nuclear transfer process. This process involves removal of the nucleus of a pluripotent cell from blastocysts followed by introduction into the enucleated cell of a nucleus extracted from an adult cell of the intended recipient of the stem cell implantation or engraftment.

A population of pluripotent or multipotent stem cells can be expanded and differentiated prior to implantation or transplantation or for other purposes using culturing methods known to the art. Among such processes can be mentioned the co-culturing of the stem cells with a feeder layer containing fibroblasts or stromal cells. Pluripotent stem cells can also be cultured in the presence of leukemia inhibitory factor (LIF). In one embodiment of the invention, HA can be included in the culture of pluripotent stem cells containing a feeder layer or bFGF or LIF in the culture of multipotent stem cells containing a cocktail of cytokines and growth factors. Methods for culturing stem cells are well known to those of ordinary skill in the art. (See e.g., Stem Cells Handbook, Small, S., (2004) Humana Press, Totoya, N.J.)

In order to demonstrate the utility of the invention, experiments using hematopoietic SCs and the hematopoietic system have been conducted and the results are set out as examples herein. It was found that HA facilitates post-chemotherapy (5-FU injection) recovery of hematopoiesis in bone marrow and shortens the time of bone marrow hypoplasia. An increased number of hematopoietic SCs and progenitor cells were indicated in the marrow of mice treated with HA. Furthermore, it was found that there was an elevated number of cells in mitosis, indicative of a higher proliferating rate. The augmentation of bone marrow hematopoiesis by HA subsequently resulted in higher numbers of mature terminally differentiated functionally active peripheral blood cells, including lymphocytes, neutrophils, monocytes, platelets and megakaryocytes, and erythrocytes in comparison with control animals. Thus, administration of HA proved effectual in fighting the 5-FU-induced bone marrow hypoplasia and pancytopenia. This suggests that HA substantially contributes to the requisite microenvironment in the bone marrow for hematopoietic SCs, which accelerates the rebound of hematopoiesis. Thus, it is believed that HA induces the balanced production of soluble and membrane-associated regulators of SC proliferation, differentiation and self-renewal. The correct composition of factors induced by HA would, therefore, help to maintain a balanced mature cell production, to avoid lineage competition and to prevent SC depletion/exhaustion.

The present invention also demonstrates that HA significantly improves chemotherapeutically perturbed hematopoiesis in mice and is therefore an appropriate therapy for treatment-induced bone marrow hypoplasia and aplasia. The use of HA will result in a better prognosis for, and more rapid recovery of, patients who undergo chemotherapy.

Since both total-body irradiation and transplantation of bone marrow cells sharply decrease the amount of HA in bone marrow (Noordegraaf et al., Exp Hematol. 1981;9:326-331), we investigated the effect of exogenous HA on SC engraftment after lethal irradiation (15.25 Gy y-rays at a dose rate of 0.85 Gy/h) followed by bone marrow transplantation. While the number of white blood cells (WBC) in control groups remained low, a complete recovery of leukocyte numbers in recipients of HA was observed on day 13. In addition, recovery of platelets (PLTs) and red blood cells (RBCs) in the peripheral blood of mice treated with HA was monitored. The enhanced recovery of peripheral blood cell counts in the HA-treated group is a result of facilitated engraftment of transplanted SCs, since analysis on bone marrow revealed an increased number of mature cells as well as their progenitors in the HA-treated group.

These findings demonstrate that HA helps restore and/or maintain a requisite microenvironmental niche in the bone marrow, which facilitates homing and engraftment of SCs.

Overall, the results presented herein demonstrate the beneficial use of HA in clinical hematology to improve bone marrow recovery after chemotherapy and body irradiation, as well as other treatment-induced damage of tissues and organs.

Modes of Administration

The HA compositions of the present invention can be administered as an aqueous solution, or they may be incorporated into carrier vehicles such as liposomes or microparticles, especially those that are targeted specifically for any tissue/organ, and administered as suspensions of these carriers. Such targeted carrier vehicles are described in the literature.

To assure more tissue-specific delivery of injected HA, it can be conjugated with a carrier that targets a particular tissue, for example, any type of SC, stromal cells, endothelium cells or other cell type to which it is desirable to direct the HA. A suitable tissue specific carrier can be, for example, a fusion protein composed of an HA-binding protein and an antibody, particularly an IgG, or antibody fragment specific for the target tissue. Suitable antibody fragments include, for example, F(ab)′ and F(ab)2′ fragments. The use of such target specific carriers will provide improved anchoring of the injected HA when treating pathological conditions associated with the loss of HA receptors.

Pharmaceutical compositions of HA can be administered intraperitoneally, intravenously, or intra-organ.

The composition of HA can be administered any time following recognition of low levels of HA in a subject.

In some applications, for example, following chemotherapy with drugs such as 5-FU under conditions that deplete, but do not eliminate stem cells, the composition of HA can be administered alone. Alternatively, the composition of HA can be combined with a suspension of SCs or, for that matter, a suspension of any other type of cell, or a tissue or organ, prior to implantation or transplantation.

In another embodiment, the composition of HA can be pre-incubated with the cellular suspension, for example, a SC suspension, prior to implantation or transplantation. In yet other embodiments HA can be administered in conjunction with therapies involving administration of colony stimulating factors such as G-CSF, GM-CSF, M-CSF, Epo, and Tpo, interleukins, stem cell factor, flt-3 ligand, or negative regulators of SC proliferation such as TGF-β, TNF-α, LIF, MIP-1α, and interferons, and other agents used in such therapies.

The HA composition can be administered once, or multiple times, so long as the subject continues to demonstrate symptoms suspected of being alleviated by HA therapy, or low levels of HA. If used in conjunction with transplanted or implanted cells, administration can be before, with or after treatment with a suspension of cells such as SCs.

The total amount of HA to be administered to a subject in each dose for a particular application that constitutes an effective therapeutic dose can be readily determined by those skilled in the art. In a preferred embodiment of the invention, the dose may be any dose between 0.1 to 100 mg/kg, and, more preferably any dose between 1 to 10 mg/kg. The term “therapeutic dose” is meant to express the amount necessary to result in an observable increase in HA levels in the subject to which the composition is administered. As such, the precise amount that represents a “therapeutic dose” can easily be determined on the basis of monitoring of the HA levels post-administration, and multiple dosing until a therapeutically effective amount has been administered.

The term “pharmaceutically acceptable” means that the carrier, diluent, excipients and salt must be compatible with the other ingredients of the formulation, and not deleterious to the recipient thereof. Pharmaceutical formulations of the present invention are prepared by procedures known in the art using well known and readily available ingredients.

“Effective amount,” “effective dose,” “effective therapeutic dose,” “therapeutically effective amount” or “pharmaceutically effective amount” means an amount of HA, in any polymorphic form, or a salt thereof that is capable of producing its intended effect. The language is intended to include an amount which is sufficient to mediate a disease or condition and prevent its further progression or ameliorate the symptoms associated with the disease or condition. Such an amount can be administered prophylactically to a patient thought to be susceptible to development of a disease or condition. Such amount when administered prophylactically to a patient can also be effective to prevent or lessen the severity of the condition. Such an amount is intended to include an amount which is sufficient to restore the microenviromnental niche, with respect to the HA content, thus addressing the impact thereon by therapy, disease or condition.

The methods using HA, and the pharmaceutically acceptable salts, solvates and hydrates thereof, have valuable pharmacological properties and can be used in pharmaceutical preparations containing the compound or pharmaceutically acceptable salts, esters or prodrugs thereof, in combination with a pharmaceutically acceptable carrier or diluent. They are useful as substances in facilitating the homing activity of stem cells to a microenvironmental niche in human or non-human animals.

Suitable pharmaceutically acceptable carriers include inert solid fillers or diluents and sterile aqueous or organic solutions. The active compound will be present in such pharmaceutical compositions in amounts sufficient to provide the desired dosage amount in the range described herein. Techniques for formulation and administration of the compounds of the instant invention can be found in Remington: the Science and Practice of Pharmacy, 19th edition, Mack Publishing Co., Easton, Pa. (1995).

For oral administration, the compound or salts thereof can be combined with a suitable solid or liquid carrier or diluent to form capsules, tablets, pills, powders, syrups, solutions, suspensions and the like. Suitable solid carriers are magnesium carbonate, magnesium stearate, talc, sugar, lactose, pectin, dextrin, starch, gelatin, tragacanth, methyl cellulose, sodium carboxymethyl cellulose, low melting waxes, and cocoa butter. In powders the carrier is a finely divided solid which is in admixture with the finely divided active ingredient. In tablets the active ingredient is mixed with a carrier having the necessary binding properties in suitable proportions and compacted in the shape and size desired. A solid carrier can be one or more substance which may also act as flavoring agents, lubricants, solubilizers, suspending agents, binders, tablet disintegrating agents and encapsulating material.

The tablets, pills, capsules, and the like may also contain a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose lactose or saccharin. When a dosage unit form is a capsule, it may contain, in addition to materials of the above type, a liquid carrier such as a fatty oil. Powders and tablets preferably contain from about 1 to about 99 weight percent of the active ingredient which is the novel compound of this invention.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. Such compositions and preparations typically contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained.

The active compounds can also be administered intranasally as, for example, liquid drops or spray. For oral or nasal inhalation, the compounds for use according to the present invention are conveniently delivered in the form of a dry powder inhaler, or an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized -aerosol the dosage unit may be determined by providing a valve to deliver a metered amount. Capsules and cartridges of gelatin for use in an inhaler or insufflator may be formulated containing a powder mix of the compound and a suitable powder base such as lactose or starch.

For parental administration the compounds useful with the present invention, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that syringability exists. It must be stable under the conditions of manufacture and storage and must be preserved against any contamination. The carrier can be solvent or dispersion medium containing, for example, water, ethanol, polyol (e.g. glycerol, propylene glycol and liquid polyethylene glycol), propylene glycol and liquid polyethylene glycol), suitable mixtures thereof, and vegetable oils. The injectable solutions prepared in this manner can then be administered intravenously, intraperitoneally, subcutaneously, or intramuscularly, with intramuscular administration being preferred in humans.

The compounds may also be formulated in rectal compositions such as suppositories or retention enemas, e.g., containing conventional suppository bases such as cocoa butter or other glycerides.

In addition, to the formulations described previously, the compounds may also be formulated as a depot preparation. Such long acting formulations may be administered by implantation, for example, subcutaneously or intramuscularly or by intramuscular injection. Thus, for example, as an emulsion in an acceptable oil, or ion exchange resins, or as sparingly soluble derivatives, for example, as sparingly soluble salts.

The effective dosage of active ingredient employed may vary depending on the particular compound employed, the mode of administration, the condition being treated and the severity of the condition being treated.

The methods and treatments mentioned herein include the above and encompass the treatment and/or prophylaxis facilitating the homing activity of stem cells to a microenvironmental niche.

The compositions are formulated and administered in the same general manner as detailed herein. The compounds of the instant invention may be used effectively alone or in combination with one or more additional active agents depending on the desired target therapy. Combination therapy includes administration of a single pharmaceutical dosage formulation which contains HA and one or more additional active agents, as well as administration of HA and each active agent in its own separate pharmaceutical dosage formulation. Where separate dosage formulations are used, HA and one or more additional active agents can be administered at essentially the same time, i.e., concurrently, or at separately staggered times, i.e., sequentially; combination therapy is understood to include all these regimens.

Preferably compounds of the invention or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient (viz., a compound of HA or salts thereof) in a unit dose of composition is a therapeutically effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

Advantageously, compositions containing the HA or the salts thereof may be provided in dosage unit form, preferably each dosage unit containing from about 0.1 to about 500 mg be administered although it will, of course, readily be understood that the amount of the compound or compounds of HA actually to be administered will be determined by a physician, in-the light of all the relevant circumstances.

### EXAMPLE 1

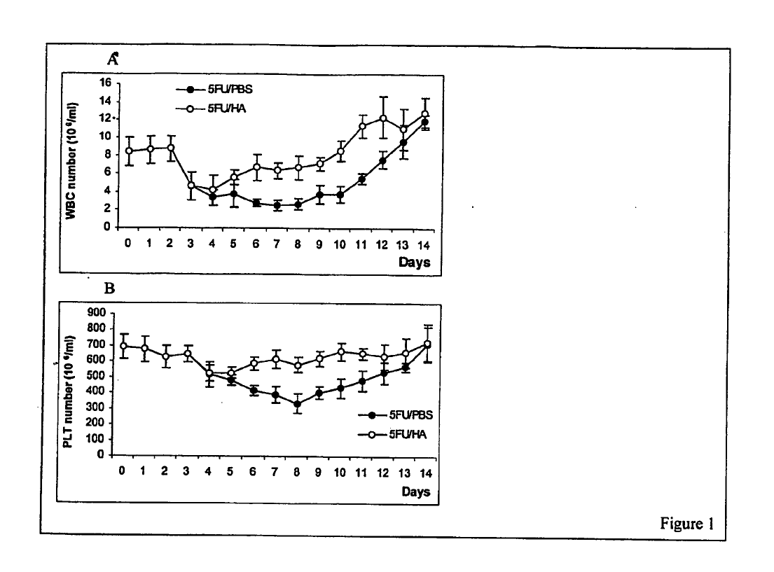

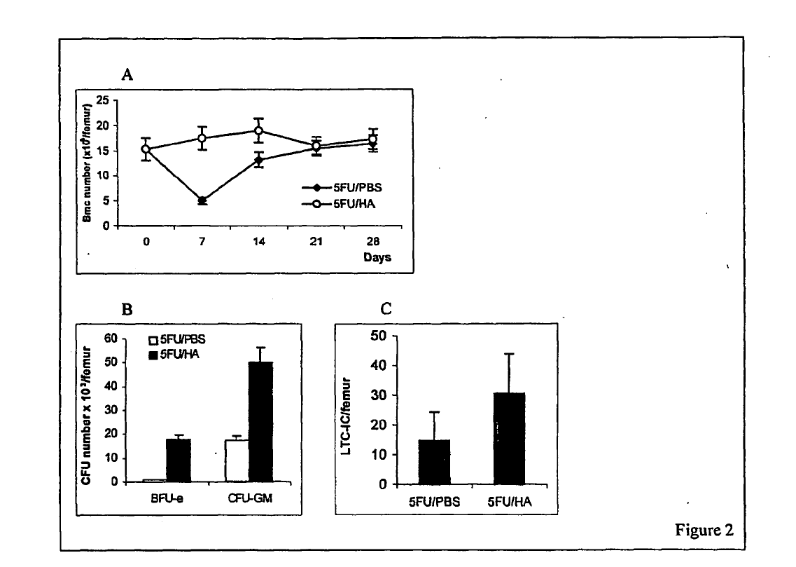

FIG. 1 demonstrates the effects of HA on recovery of peripheral blood cells after 5-FU administration. 5-FU was intraperitoneally injected in mice at 150 mg/kg. The counts of white blood cells (WBC), red blood cells (RBC), platelets (PLT), hemoglobin (HGB) and hematocrit (HCT) were monitored daily for two weeks. As expected, the treatment of mice with 5-FU induced severe bone marrow hypoplasia and pancytopenia. The numbers of WBC and PLT dropped from 8.4±1.5×10⁶/ml and 678.4±82×10⁶/ml before 5-FU administration to 2.52±0.5×10⁶/ml and 388±50×10⁶/ml, respectively, 7 days later (FIGS. 1A, B). The total number of mononuclear cells in the bone marrow decreased from 15.3±2.2×10⁶/femur before to 5.00±0.65×10⁶/femur 7 days after 5-FU administration (FIG. 2A). All parameters were recovered to normal in 14 days after 5-FU administration. To examine the effect of HA on 5-FU-perturbed hematopoiesis, 5-FU-treated (day 0) mice were administered 100 μg/mouse HA as a 0.05% solution in PBS (HA from Sigma-Aldrich, Catalog No.: H1751) on days 4, 6, 10, and 13. A control group of animals was treated with a 200-μl injection of PBS. The peripheral blood from HA- and PBS-treated mice was collected daily and examined for numbers of WBC, RBC, PLT, HGB, and HCT. The numbers of WBC in HA-treated mice were significantly higher starting from day 5 (2-2.5 fold) as compared to the control PBS-treated group (FIG. 1A). To demonstrate a dose-dependent effect of HA on WBC recovery, mice were administered with various doses of HA (0-1000 μg/mouse), and the peripheral blood samples were evaluated for the leukocyte number on day 7. The most effective amount of HA was found to be 100 μg/mouse (or 3 mg/kg). The number of PLT in HA-treated mice was increased starting from day 5 and was elevated by a factor of 1.7 on day 8 (FIG. 1B). From week 2 the parameters observed in the HA-treated group corresponded to those in normal mice prior to 5-FU treatment. Thus, administration of HA rescued mice from 5-FU-induced leukocytopenia and thrombocytopenia.

Because mature blood cells are a product of proliferation and differentiation of hematopoietic progenitors, we next examined the effect of HA on bone marrow hematopoiesis in 5-FU-treated mice. 5-FU-treated mice (day 0) were administered PBS or 100 μg/mouse HA (as a 0.05%solution in PBS) on day 4, 6, 10, and 13. Bone marrow cells were harvested on days 7, 14, 21, and 28. On day 7 the total number of bone marrow cells in the HA-treated mice was 3.5-fold higher in comparison to the PBS-treated mice (FIG. 2A). To evaluate the effect of HA on different hematopoietic lineages, a morphological analysis of the bone marrow cells was performed as followed. Smears of bone marrow cells were fixed on glass slides in methanol at −20° C. for 20 min and dried at room temperature, the slides were incubated with Filipson's dye (25% Giemsa dye in 96% ethanol) for 15 min, extensively washed with distilled water (pH 7.0), dried, and covered with a cover slip. Examination of the slides under the microscope revealed that the numbers of mature myeloid and lymphoid elements in the bone marrow were increased in the mice that received HA treatment following 5-FU administration (Table 1).

TABLE 1
Effect of HA on bone marrow cell counts after 5-FU administration

| | non- | | | | | | |
|---|---|---|---|---|---|---|---|
| | treated | control | | | HA | | |
| Cell types: | Day 0 | Day 7 | Day 14 | Day 21 | Day 7 | Day 14 | Day 21 |
| promyelocyte | 0.3 | 0.2 | 1.5 | 0.1 | 0.8 | 1 | 0.3 |
| melocyte | 3 | 5.4 | 4 | 3 | 4.2 | 6 | 4.7 |
| metamyelocyte | 3.7 | 4 | 6 | 5.3 | 4.3 | 5 | 5.7 |
| band-neutrophil | 17 | 4.5 | 18 | 15 | 13 | 25 | 18.3 |
| segmented neutrophil | 27.3 | 2 | 22 | 39 | 17.6 | 19 | 49 |
| eosinophil | 1 | 0 | 0.5 | 0.3 | 0.6 | 0 | 0.3 |
| monocyte | 0.7 | 0.5 | 2 | 2 | 1 | 2.5 | 2.8 |
| lymphocyte | 21.3 | 11.1 | 6.5 | 13.3 | 33 | 20.3 | 29.7 |
| basophilic normocyte | 4.5 | 3.5 | 2.5 | 1 | 5 | 2 | 2 |
| oxyphilic normocyte | 0.3 | 0.4 | 0.5 | 0.3 | 0.5 | 0.3 | 0.5 |
| polychromatophilic | 28.2 | 17.2 | 20 | 19.6 | 17.9 | 19 | 21.2 |
| normocyte | | | | | | | |
| plasma cell | 0.5 | 1 | 0.5 | 0.7 | 1.5 | 0.4 | 0.5 |
| platelet/field | 7 | 2 | 2 | 5 | 25 | 20 | 10 |

Interestingly, the animals treated with HA also showed an increased number of bone marrow cells in mitosis (1.7/100) as compared to control (0.3/100). These data are suggestive of a higher number of proliferating progenitor cells in the bone marrow of HA-treated mice. To examine this assumption, bone marrow cells were cultured in methylcellulose to evaluate the number of proliferating lineage-committed progenitors. Bone marrow cells were harvested and plated at a concentration of 1×10⁴ cells/ml in 24-well plates in semisolid methylcellulose containing 30% FCS, 1% BSA, 104 M 2-mercaptoethanol, 2 mM L-glutamine (StemCell Technologies, Vancouver, Canada). Conditioned medium from WEHI-3B was added (15% v/v) as a source of interleukin-3. The cultures were incubated at 37° C. in a humidified atmosphere of 5% CO₂. Colonies containing more then 20 cells were counted under the inverted microscope after 7 days of culture. To culture erythroid burst-forming units (BFU-e), 10 U/ml of erythropoietin (Boehringer-Mannheim, Germany) was added. Colonies containing at least 500 cells were counted after 14 days. The number of myeloid progenitors in the mice treated with HA was 2.9-fold higher, and the number of early erythroid progenitors was 21.5-fold higher as compared to control (FIG. 2B). The number of megakaryocytes in the bone marrow of HA-treated mice showed a 3.7-fold increase. We next investigated whether HA stimulation benefited the pool of committed progenitors at the cost of damaging more primitive progenitors that were measured by using a long-term culture-initiating cells (LTC-IC) assay. The number of LTC-IC in the bone marrow of mice treated with HA was evaluated using a limited dilution assay. We monitored a trend in the increase in number of LTC-IC in the bone marrow of 5FU/HA vs. 5FU/PBS mice. Although the difference was not statistically significant (p>0.1), the number of LTC-IC was elevated from 14.8±9.6/femur in control to 30.6±13.3/femur in HA-treated animals (FIG. 2C). These findings suggest that the increase in the number of mature cells and committed progenitors in the bone marrow of HA-treated mice does not result in the exhaustion of the pool of more primitive stem cells as measure by LTC-IC. Thus, HA promoted bone marrow hematopoietic activity, which had been impaired by 5-FU.

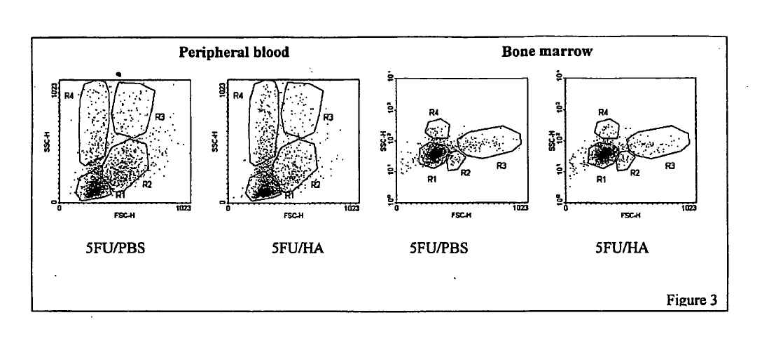

Since the mobilization effect of HA on hematopoietic cells in healthy individuals has been demonstrated using HA having a molecular weight range from 178 kD to 562 kD (L. Pilarski, Canadian patent application 2,199,756), we further tested the effect of HA on cell mobilization in 5-FU treated mice. Mice were administered 150 mg/kg 5FU followed by HA as a 0.05% solution in PBS or PBS infusion (3 mice per group) on day 4. Twelve and 24 hours after the HA infused mice were sacrificed, peripheral blood (PB) or bone marrow (BM) cells were harvested and analyzed on a flow cytometer. Each sample was measured for 10⁴ total events (100%). Size (as X-Mean), granularity (as Y-Mean) and the percentage of the total cells for each cell population was evaluated and expressed as mean ±SD. (See Table 2 and FIG. 3.) FACS analysis revealed that neither after 12 hours (data not shown) nor 24 hours (FIG. 3) was the composition of cell populations in the peripheral blood-or bone marrow changed. It can be concluded, therefore, that the mobilization of cells from the bone marrow to the peripheral blood is not induced by HA under these conditions. We anticipate several possible reasons for this observation: 1. Chemotherapy might affect the expression of HA receptors, resulting in an impaired mobilization response to HA treatment; 2. Chemotherapy induces hypoplasia of bone marrow and therefore eliminates cellular resources for detectable mobilization; 3. mobilization is activated by HA having a limited molecular weight range for each polymer, perhaps by targeting different HA receptors/isoforms. Whatever the reason, this application is neither limited by nor dependent on the biological mechanism of stem cell mobilization.

TABLE 2
Analysis of the peripheral blood (PB) and bone marrow
(BM) cell composition in 5FU-administered mice 24
hours after HA administration

| PBS | | | HA | | |
|---|---|---|---|---|---|
| X-Mean | Y-Mean | % Total | X-Mean | Y-Mean | % Total |

Population
PB

| | | | | | | |
|---|---|---|---|---|---|---|
| R1 | 289.8 ± 2.9 | 120.7 ± 0.85 | 56.4 ± 5.8 | 291.4 ± 0.9 | 117.7 ± 1.7 | 60.14 ± 8.1 |
| R2 | 485.9 ± 10.3 | 270.7 ± 9.6 | 17.8 ± 4.2 | 471.4 ± 10 | 262 ± 6.9 | 13.6 ± 3.8 |
| R3 | 543.9 ± 13.7 | 738 ± 0.9 | 2.8 ± 0.9 | 535.3 ± 12.8 | 746.8 ± 10.2 | 2.3 ± 1.2 |
| R4 | 293 ± 2.5 | 572.4 ± 15.8 | 9.7 ± 1.2 | 291.2 ± 4.6 | 587.7 ± 2.2 | 11.23 ± 0.96 |
| Population | | | | | | |
| BM | | | | | | |
| R1 | 305.3 ± 0.4 | 38.5 ± 0.3 | 85.5 ± 0.8 | 301.3 ± 0.6 | 40.8 ± 0.1 | 87.2 ± 0.64 |
| R2 | 463.3 ± 2.3 | 27.7 ± 0.3 | 1.2 ± 0.23 | 470.2 ± 2.3 | 27.7 ± 0.6 | 1.5 ± 0.12 |
| R3 | 642.6 ± 3 | 74.9 ± 0.7 | 4.1 ± 0.47 | 653.2 ± 4 | 78.1 ± 1.88 | 3.4 ± 0.22 |
| R4 | 313.6 ± 1.7 | 185 ± 1.4 | 2 ± 0.29 | 310.8 ± 3.5 | 191.7 ± 4 | 1.43 ± 0.16 |

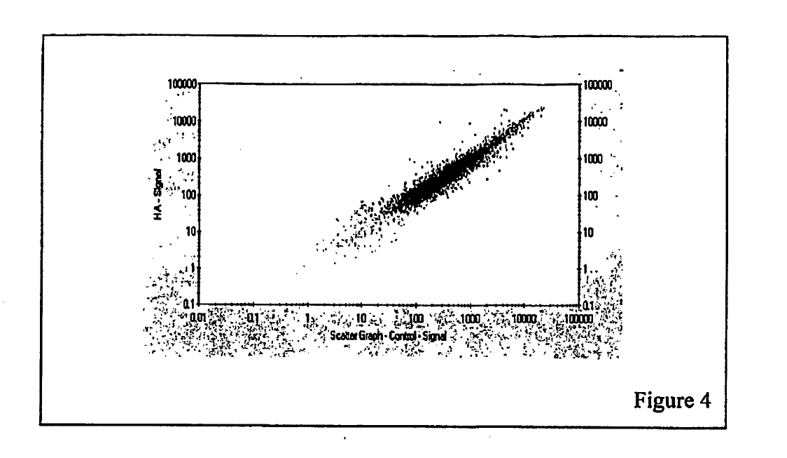

We have previously demonstrated that HA does not promote proliferation of hematopoietic progenitors directly (Khaldoyanidi, S., et al. Blood. (1999) 94:940-949). Subsequent studies demonstrated that HA up-regulates the production of hematopoiesis-supporting cytokines IL-1 and IL-6 by the cells of the bone marrow hematopoietic microenvironment. However, the experiment with IL-1 and IL-6 neutralizing antibodies suggested that in addition to IL-1 and IL-6, other hematopoiesis-supportive soluble factors are produced by the HA stimulated hematopoietic microenvironment. To identify other molecules that mediate HA effects, we performed gene expression profiling using Affymetrix chip technology. Mice were administered 150mg/kg 5FU at day 1, followed by the infusion of 100 μg HA as a 0.05% solution in PBS at day 4. Twenty hours later the animals were sacrificed, the bone marrow harvested and total RNA isolated using a Qiagen RNA isolation kit. Probe preparation and chip hybridization was performed according to the manufacturer's recommendations (Affymetrix, Alameda). Differentially expressed genes were analyzed with the Affymetrix Data Mining Tool software. In this log transformed graph the hybridization signals for over 10,000 genes are plotted. Hybridization signals obtained from control samples (5FU/PBS) (X-axis) are compared to samples from mice treated with HA (5FU/HA) (Y-axis). Genes that were statistically significantly detected in the samples are represented by black spots at higher hybridization intensities (top right of the data trend in FIG. 4). Non-detected genes are shown by gray spots. Spots that deviate from the main trend in the plot are differentially expressed between the two samples. We identified a total of 179 genes that were called as highly significantly differentially expressed in the bone marrow of HA-treated mice (5FU/HA) vs. control mice (5FU/PBS). A replicate from a separately treated mouse gave almost completely concordant data. The differentially expressed genes could be grouped as follows: (1) Transcription regulation, hormone receptors and DNA replication factors; (2) Signal transduction cascade regulators; (3) Apoptosis regulation; (4) Migration mediating enzymes; (5) Cell surface associated molecules; (6) Soluble factors.

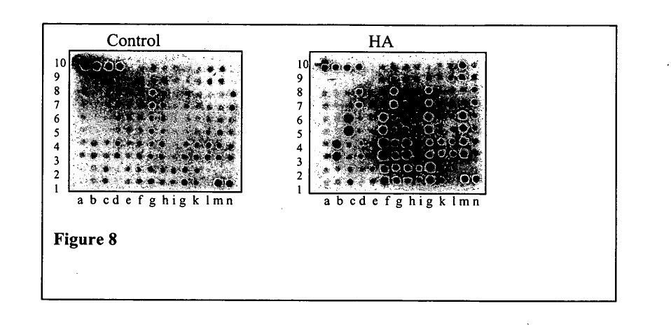

A further experiment was performed to determine the effect that HA has on cytokine production in long-term bone marrow cultures (LTBMC). Murine myeloid LTBMC were initiated as follows: freshly isolated bone marrow cells (10⁶ cells/ml) were cultured in Dulbecco modified Eagle medium (DMEM) supplemented with 20% horse serum (StemCell Technologies, Vancouver, Canada) and 10-6 M hydrocortisone (Sigma, St. Louis, Mo.) in 6-well plates at 37° C. in a humid atmosphere containing 5% CO₂. Cultures were fed weekly by changing half of the culture medium. On week 4, the non adherent cells were removed, the adherent layers were carefully washed and incubated without (control) or with 100 μg/ml HA in serum free DMEM for 24 hours. Thereafter, supernatants were collected and assayed using protein array technology (Ray Bio Mouse Cytokine Antibody Array III & 3.1 (62), Ray Biotech Inc. Atlanta, Ga., USA) according to manufacture's recommendations. HA up-regulated the expression of MCP-1, IL-6, G-CSF, IL-12, MIP-1α, TNF-α, MIP-1γ, IGFBP-3, IL-1, KC, VCAM-1, CXCL-16, LIX, and RANTES (FIG. 8 and Table 3). Overall, our results suggest that HA is a biologically active component of microenvironment and is involved in regulating the expression of genes and their products which mediate stem cell behavior.

TABLE 3
is a well by well indication of the cytokine assayed in each well.

| | | | | | | | | | | | | | |
|---|---|---|---|---|---|---|---|---|---|---|---|---|---|
| Pos | Pos | Pos | Pos | Blank | Axl | BLC | CD30L | CD30T | CD40 | CRG-2 | CTACK | CXCL16 | Eotaxin |
| Neg | Neg | Neg | Neg | Blank | Axl | BLC | CD30L | CD30T | CD40 | CRG-2 | CTACK | CXCL16 | Eotaxin |
| Eotax-2 | Fas | Frac | GCSF | GMCSF | IFNγ | IGFBP-3 | IGFBP-5 | IGFBP-6 | IL-1α | IL- | IL-2 | IL-3 | IL- |
| | Lig | talk | | | | | | | | 1β | | | 3Rb |
| Eotax-2 | Fas | Frac | GCSF | GMCSF | IFNγ | IGFBP-3 | IGFBP-5 | IGFBP-6 | IL-1α | IL- | IL-2 | IL-3 | IL- |
| | Lig | talk | | | | | | | | 1β | | | 3Rb |
| IL-4 | IL-5 | IL-6 | IL-9 | IL- | IL- | IL-12 | IL-13 | IL-17 | KC | Leptin R | Leptin | LIX | L- |
| | | | | 10 | 12 | p70 | | | | | | | Selectin |
| | | | | | p40 | | | | | | | | |
| IL-4 | IL-5 | IL-6 | IL-9 | IL- | IL- | IL-12 | IL-13 | IL-17 | KC | Leptin R | Leptin | LIX | L- |
| | | | | 10 | 12 | p70 | | | | | | | Selectin |
| | | | | | p40 | | | | | | | | |
| Lymphot | MCP1 | MCP-5 | MCSF | MIG | MIP- | MIP- | MIP-2 | MIP- | MIP- | PF-4 | P- | RANTES | SCF |
| | | | | | 1α | 1γ | | 3β | 3α | | Selectin | | |
| Lymphot | MCP1 | MCP-5 | MCSF | MIG | MIP- | MIP- | MIP-2 | MIP- | MIP- | PF-4 | P- | RANTES | SCF |
| | | | | | 1α | 1γ | | 3β | 3α | | Selectin | | |
| SDF- | TARC | TCA-3 | TECK | TIMP-1 | TNFα | sTNFRI | sTNFRII | TPO | VCAM-1 | VEGF | Blank | Blank | Blank |
| 1α | | | | | | | | | | | | | |
| SDF- | TARC | TCA-3 | TECK | TIMP-1 | TNFα | sTNFRI | sTNFRII | TPO | VCAM-1 | VEGF | Blank | Pos | Pos |
| 1α | | | | | | | | | | | | | |

### EXAMPLE 2

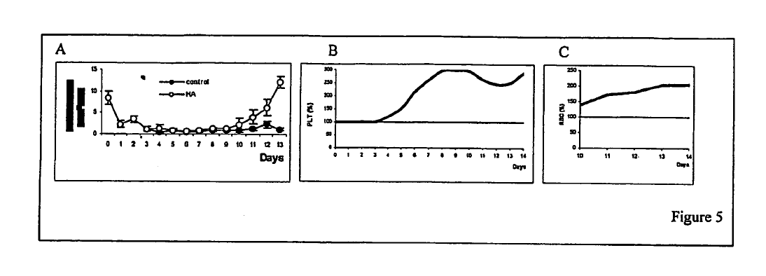

Total-body irradiation sharply decreases the amount of GAGs, including HA, in the spleen and bone marrow. Furthermore, transplantation of bone marrow cells results in a second relapse of HA concentration in hematopoietic tissue. Thus, we investigated the effect of HA on the peripheral blood and bone marrow cell recovery after total body irradiation followed by bone marrow transplantation. Recipient mice were lethally (15.25 Gy at a dose rate of 0.85 Gy/h) irradiated to eliminate endogenous bone marrow hematopoiesis. Hematopoietic Stem/Progenitor Cells (HSPC) were obtained from donor mice, pretreated with 5-FU (150 mg/kg body weight) to eliminate the proliferating committed progenitor cell pool, and transplanted into the recipient mice (10⁴ cells/mouse) 24 hours after irradiation. The recipient mice were administered 200 μl/mouse PBS (control group) or 100 μg/mouse of HA as a 0.05% solution in PBS (Sigma-Aldrich) on day 4, 6, 10, and 13 after transplantation. The number of peripheral WBC was measured daily. A significant increase in WBC number in the peripheral blood of mice treated with HA was detected on day 11 (p<0.0 1) as compared to control. While the number of WBC in control groups remained low, a complete recovery of leukocyte numbers in HA-treated recipients was observed on day 13 (FIG. 5A). In addition, facilitated recovery of PLT and RBC in the peripheral blood of HA-treated mice was monitored. The numbers of PLT and RBC were elevated 3-fold and 2-fold, respectively in the HA-treated group. PLT and RBC levels are shown as a percent increase, where counts from control PBS administered group are taken as 100% (FIGS. 5B and 5C). The increased number of RBC correlated with greater values of HCT and HGB in mice administered HA in comparison with the control mice.

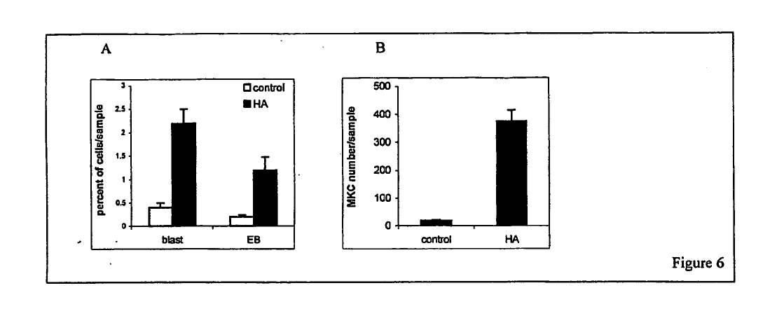

To demonstrate that the enhanced recovery of peripheral blood cell counts in mice treated with HA is a result of facilitated engraftment of transplanted HSPC, we further evaluated hematopoietic activity in the bone marrow. Seven days after the transplantation of HSPC, the bone marrow was harvested and examined. A morphological analysis of the bone marrow revealed that the total number of blast cells in the group treated with HA was 10 times higher (FIG. 6A) than in the PBS-treated control group. In addition, a 3-fold increase in the number of erythroblasts (FIG. 6A) and an 18.8-fold increase in megakaryocyte count was observed in the bone marrow of the HA-treated mice in comparison to control (FIG. 6B). The elevated number of megakaryocytes correlated with higher numbers of platelets in the bone marrow of the HA-treated mice: the platelet count in these mice was 20±1.5/field, while in the control group it remained 4±0.2/field.

Thus, we have demonstrated that HA provides more favorable conditions for engraftment of SC and subsequently tissue recovery/remodeling.

We claim:

1. A method of treating a pathological condition in a subject that is associated with decreased levels of hyaluronic acid in a microenviromnental niche of a tissue or organ comprising administration to the subject of an effective dose of hyaluronic acid, or a pharmaceutically acceptable salt thereof to facilitate stem cell homing.

2. A method according to claim 1 wherein the effective dose is from 0.1 to 100 mg/kg.

3. A method according to claim 2 wherein the effective dose is from 1 to 10 mg/kg.

4. A method according to claim 1 wherein the dose is administered intraperitoneally, intravenously or intraorgan.

5. A method according to claim 1 wherein the hyaluronic acid is incorporated into a carrier vehicle.

6. A method according to claim 5 wherein the carrier vehicle is a liposome or microparticle.

7. A method according to claim 5 wherein the hyaluronic acid is conjugated with a tissue specific carrier.

8. A method according to claim 7 wherein the tissue specific carrier comprises a fusion protein of HA binding protein and a F(ab)2 or F(ab) fragment directed against a tissue specific cell surface antigen.

9. A method according to claim 1 wherein the hyaluronic acid is administered with an effective amount of an agent selected from the group consisting of positive or negative regulators of stem and committed progenitor cell proliferation, chemokines and SDF-1.

10. A method according to claim 1 wherein the hyaluronic acid has a molecular weight between the range of about 15,000 daltons to about 2,000,000 daltons.

11. A method according to claim 10 wherein the hyaluronic acid has a molecular weight between the range of about 100,000 daltons to about 1,500,000 daltons.

12. A method according to claim 10 wherein the hyaluronic acid has a molecular weight between the range of about 500,000 daltons to about 1,000,000 daltons.

13. A method according to claim 10 wherein the hyaluronic acid has a molecular weight between the range of about 575,000 daltons to about 900,000 daltons.

14. A method according to claim 1 wherein the hyaluronic acid is a mixture of hyaluronic acid polymers having an average molecular weight between the range of about 750,000 daltons to about 2,000,000 daltons.

15. A method according to claim 14 wherein the hyaluronic acid is a mixture of hyaluronic acid polymers having an average molecular weight of about 750,000 daltons.

16. A method according to claim 1 wherein the hyaluronic acid is derived from a source comprising eukaryotic source, prokaryotic source, animal source, mammalian source, fowl source, bacterial source, fungal source, synthetic source, recombinant source, recombinant hyaluronic acid synthase cell line source, umbilical cord source, nasal source, rooster comb source, streptomyces source, streptococcal source or combinations thereof.

17. A method according to claim 16 wherein the hyaluronic acid is derived from umbilical cord source.

18. A method according to claim 1 wherein the administration of hyaluronic acid is to facilitate hematopoiesis following a therapy that decreases the levels of hyaluronic acid in the microenvironmental niche.

19. A method according to claim 1 wherein the subject to whom hyaluronic acid is administered exhibits a condition selected from the group consisting of pancytopenia, neutropenia, thrombocytopenia, anemia, lymphocytopenia or any combination or subcombination thereof.

20. A method according to claim 1 wherein the condition is the result of a therapy or a disease that decreases the levels of hyaluronic acid at the microenvironmental niche.

21. A method according to claim 20 wherein the therapy is chemotherapy, radiotherapy or hormonal therapy.

22. A method according to claim 20 wherein the therapy is cytotoxic therapy.

23. A method for treating a subject to improve the engraftment of implanted or transplanted stem cells comprising the administration to the subject of an effective dose of hyaluronic acid or a pharmaceutically acceptable salt thereof.

24. A method according to claim 23 wherein the stem cells are selected from the group consisting of totipotent stem cells, pluripotent stem cells, multipotent stem cells and combinations thereof.

25. A method according to claim 24 wherein the stem cells are multipotent stem cells.

26. A method according to claim 25- wherein the multipotent stem cells are obtained by causing the differentiation of totipotent or pluripotent stem cells.

27. A method according to claim 26 wherein the pluripotent stem cells are manipulated by the nuclear transfer process.

28. A method according to claim 25 wherein the multipotent stem cells are selected from the group consisting of hematopoietic, neuronal, mesenchymal, epithelial, endothelial, pancreatic hepatic, adult stem cells and combinations thereof.

29. A method according to claim 23 wherein the stem cells are selected from the group of stem cells consisting of bone marrow stem cells, peripheral blood stem cells, umbilical cord blood stem cells, brain stem cells, pancreas stem cells, liver stem cells, mucosal tissue stem cells, skin stem cells and combinations thereof.

30. A method according to claim 23 wherein the stem cells are primary stem cells isolated from the tissue of a living donor of a cadaver or stem cells cultured in in vitro stem cell culturing conditions.

31. A method according to claim 30 wherein the stem cells are cultured in vitro in a medium employing a feeder layer of fibroblasts or stromal cells and the medium contains hyaluronic acid.

32. A method according to claim 30 wherein the stem cells are pluripotent stem cells cultured in vitro in a culture medium that contains LIF and hyaluronic acid.

33. A method according to claim 23 wherein the stem cells are hematopoietic stem cells.

34. A method according to claims 23 wherein mesenchymal stem cells are coadministered with hematopoietic stem cells.

35. A method according to claim 23 wherein the stem cells are mesenchymal stem cells.

36. A method according to claim 35 wherein the mesenchymal stem cells are administered directly to tissue, the tissue being selected from the group of tissues comprising bone/bone marrow tissue, cartilage tissue, muscle tissue, tendon tissue, and brain tissue; wherein the tissues are administered alone or in combination with other stem cells.

37. A method according to claim 23 wherein the subject is implanted or transplanted with stem cells to treat a pathological condition.

38. A method according to claim 37 wherein the stem cells are pancreatic stem cells and the pathological condition is diabetes.

39. A method according to claim 37 wherein the pathological condition is heart damage.

40. A method according to claim 39 wherein the heart damage is the result of an infarct or surgery.

41. A method according to claim 23 wherein the hyaluronic acid is administered before, with or after the implantation or transplantation of the stem cells.

42. A method according to claim 23 wherein the subject receives therapy prior to transplantation of the stem cells.

43. A method according to claim 42 wherein the therapy is cytotoxic therapy.

44. A method according to claim 42 wherein the therapy comprises chemotherapy, radiotherapy or hormonal therapy.

45. A method according to claim 42 wherein the therapy is ablative therapy.

46. A method according to claim 23, wherein the dose is from 0.1 to 100 mg/kg.

47. A method according to claim 46 wherein the dose is from 1 to 10 mg/kg.

48. A method according to claim 23 wherein the dose is administered intravenously, intraperitoneally or intraorgan.

49. A method according to claim 23 wherein the hyaluronic acid has a molecular weight between the range of about 15,000 daltons to about 2,000,000 daltons.

50. A method according to claim 49 wherein the hyaluronic acid has a molecular weight between the range of about 100,000 daltons to about 1,500,000 daltons.

51. A method according to claim 49 wherein the hyaluronic acid has a molecular weight between the range of about 500,000 daltons to about 1,000,000 daltons.

52. A method according to claim 49 wherein the hyaluronic acid has a molecular weight between the range of about 575,000 daltons to about 900,000 daltons.

53. A method according to claim 23 wherein the hyaluronic acid is a mixture of hyaluronic acid polymers having an average molecular weight between the range of about 750,000 daltons to about 2,000,000 daltons.

54. A method according to claim 53 wherein the hyaluronic acid is a mixture of hyaluronic acid polymers having an average molecular weight of about 750,000 daltons.

55. A method according to claim 23 wherein the hyaluronic acid is derived from a source comprising eukaryotic source, prokaryotic source, animal source, mammalian source, fowl source, bacterial source, fungal source, synthetic source, recombinant source, recombinant hyaluronic acid synthase cell line source, umbilical cord source, nasal source, rooster comb source, streptomyces source, streptococcal source or combinations thereof.

56. A method according to claim 55 wherein the hyaluronic acid is derived from umbilical cord source.

57. In a method of treating a subject with a drug or radiation that results in bone marrow dysfunction, the improvement comprising treating the subject having the bone marrow dysfunction with an effective amount of hyaluronic acid, or a pharmaceutically acceptable salt thereof to restore the microenvironmental niche for stem cell homing.

58. A method for improving the recovery of the number of stem cells and their functions in a subject having a depleted population of stem cells as the result of a pathological condition or of a therapy which depletes the stem cell population or proper function of stem cells comprising administering to the patient an effective amount of hyaluronic acid, or a pharmaceutically acceptable salt thereof to facilitate homing of the stem cells.

59. A method according to claim 58 wherein the stem cells are multipotent stem cells.

60. A method according to claim 59 wherein the multipotent stem cells are selected from the group consisting of hematopoietic stem cells, neuronal stem cells, mesenchymal stem cells, epithelial stem cells, endothelial stem cells, liver stem cells, hepatic stem cells, pancreatic stem cells, adult stem cells and combinations thereof.

61. A method according to claim 59 wherein the multipotent stem cells are obtained by causing the differentiation of totipotent or pluripotent stem cells.

62. A method according to claim 59 wherein the pluripotent stem cells are manipulated by a nuclear transfer process.

63. A method according to claim 58 wherein the stem cells are depleted by therapy.

64. A method according to claim 63 wherein the population of stem cells are hematopoietic stem cells.

65. A method according to claim 63 wherein the therapy is cytotoxic therapy.

66. A method according to claim 63 wherein the therapy is chemotherapy, radiotherapy or hormonal therapy.

67. A method according to claim 58 wherein the effective amount is a dose of 0.1 to 100 mg/kg.

68. A method according to claim 67 wherein the dose is from 1 to 10 mg/kg.

69. A method for culturing stem cells, the improvement comprising including hyaluronic acid in the culture medium to facilitate homing of the cultured cells to a microenvironmental niche.

70. A method according to claim 69 wherein the culture conditions employ a feeder layer of fibroblasts or stromal cells.

71. A method according to claim 69 wherein the stem cells are pluripotent or multipotent stem cells.

72. A method according to claim 71 wherein the stem cells are pluripotent stem cells and the culture medium contains LIF.

73. A method according to claim 69 wherein the cells are cultured with hyaluronic acid to facilitate homing to a microenvironmental niche following transplant or implant into a patient.

74. A method for facilitating homing activity of stem cells in a subject comprising the step of providing hyaluronic acid wherein the hyaluronic acid functions to home stem cells to a microenviromnental niche.

75. The method of claim 74 wherein the hyaluronic acid is administered as a combination therapy.

76. The method of claim 75 wherein the hyaluronic acid is administered as a combination therapy with an agent comprising positive regulators of stem cell proliferation, negative regulators of stem cells proliferation, positive regulators of committed progenitor cell proliferation, negative regulators of committed progenitor cell proliferation, cytokines, chemokines or SDF-1.

77. The method according to claim 76 wherein the hyaluronic acid is used in a form conjugated with tissue specific carrier, which is a fusion protein consisting of hyaluronic acid binding protein fused to an F(ab)2 or F(ab) fragment directed against a tissue specific cell surface antigen.

78. The method according to claim 74 wherein the hyaluronic acid is provided to the stem cells ex vivo and then implanted or transplanted into a subject.
